(19) United States
(12) Patent Application Publication
(10) Pub. No.: US 2024/0280521 A1
(43) Pub. Date: Aug. 22, 2024

(54) CRYSTALLINE RESIN CONTAINING MICROSCOPIC SURFACE RELIEFS AND METHODS AND SYSTEMS FOR GENERATING THE SAME

(71) Applicant: Ori Lieberman, San Diego, CA (US)

(72) Inventor: Ori Lieberman, San Diego, CA (US)

(21) Appl. No.: 18/571,410

(22) PCT Filed: Jun. 9, 2022
(86) PCT No.: PCT/US2022/032825

§ 371 Date: Dec. 18, 2023

Related U.S. Application Data
(60) Provisional application No. 63/212,437, filed on Jun. 18, 2021.

(51) Int. Cl. G01N 23/2251; G03H 1/00; G03H 1/02

(52) U.S. Cl. CPC G01N 23/2251; G03H 1/0011; G03H 1/0244; G01N 2223/102; G01N 2223/418; G01N 2223/611

(57) ABSTRACT

An article comprising a crystalized resin material, the material having microscopic surface reliefs in a surface thereof, wherein the surface reliefs form optical structures which generate an optical image and or holographic information, the crystalized resin material is a cannabis derived resin and the microscopic surface reliefs formed in the resin form optical structures which generate an optical image or optical information.

### CROSS-REFERENCE TO RELATED APPLICATION

This application claims priority to U.S. Provisional Patent Application Ser. No. 63/212,437 titled “Crystalline Resin Containing Microscopic Surface Reliefs And Methods And Systems For Generating The Same”, filed Jun. 18, 2021, the disclosure of which is incorporated by reference herein in its entirety.

### FIELD OF THE INVENTION

This invention generally relates to crystalline resins that have microscopic surface reliefs and methods and systems for generating the same. More particularly, the resin is a cannabis-derived resin and may be made mostly of isolated cannabinoids, such as THCA (tetrahydrocannabinol acid extracted from trichomes), CBDA (cannabidiolic acid), CBGA (cannabigerolic acid), or any other cannabinoid whose physical and chemical make-up allows the cannabis-derived resin to crystallize or harden. More particularly, the surface reliefs may generate an optical image, such as a hologram, or generate data or codes, in the crystalline resin that can be useful for aesthetic purposes as well as for security purposes.

### BACKGROUND

Cannabis-derived resins are sold in a variety of forms and for a variety of purposes. Some cannabis-derived resins may contain cannabinoids of a sufficient purity that allows the resin to crystallize. Such cannabis-derived resins may be heated and ingested or consumed through vaporization or by smoking. They may also be made into an edible product and consumed orally.

An advantage of crystalline cannabis-derived resins (i.e., a cannabis-derived resin that has crystallized) is that they typically have a longer shelf life than cannabis-derived resins that are not crystallized. A crystalline resin product does not rapidly degrade in quality, taste or THC or CBD content over time or with exposure to moisture or temperature changes. Many other forms of cannabis products or derivatives degrade with exposure to oxygen, moisture or heat, causing consumers a subdued, diluted or stale cannabis experience. For example, cannabis products commonly contain terpenes, flavonoids, THC, and other cannabinoids which evaporate, degrade or transform with exposure to oxygen, heat, moisture and UV light.

A need has arisen in the cannabis industry for the capability of producing such crystalline cannabis-derived resins that have visual or optical effects and designs on them. Such effects and designs may be used for marketing purposes or for security purposes, for example to verify the source of a product is authentic.

The effects and designs are desired to add to a product's appearance and may aid in branding or marketing such products. Alternately, or in addition, a need has arisen for the capability of providing security such as through “track and trace” programs, to allow cannabis producers to identify their products and prevent counterfeiting or product tampering.

### SUMMARY

One aspect of the invention includes a crystalline cannabis-derived resin that has microscopic surface reliefs that form optical structures. The surface reliefs and optical structures may be used for branding or marketing visually appealing products having images therein. The reliefs and optical structures may also form data or codes that may act as a security features that can help prevent counterfeiting and help authentication of a product's source, such as in a “track and trace” program.

In another aspect, a method for fabricating an optical image in a crystalline cannabis-derived resin, the method comprising, the steps of obtaining a cannabis-derived resin that can be crystalized, heating the resin, placing the heated resin onto a production tool that has a surface with microscopic surface reliefs, such as a shim, film or mold, spreading the heated resin over at least part of the surface of the production tool, imprinting surface reliefs in the heated resin, allowing the resin to cool and crystallize and removing the resin from the production tool after it has crystallized sufficiently. The microscopic surface reliefs formed in the resin form optical structures which generate an optical image.

In yet another aspect, the optical structures include one or more of the following: a grating, a hologram, a kinegram, a Fresnel lens, a diffractive optically variable image device, a pixelgram, a holographic stereogram, a diffraction identification device, a photonic structure, a dielectric structure, a volume hologram, an interference security image structure, a computer-generated hologram, or an electron-beam grating.

In another aspect, the cannabis derived resin has cannabinoids selected from the group of THCA (tetrahydrocannabinol acid extracted from trichomes), CBDA and CBGA.

In another aspect of the invention there is an article comprising a crystalline resin material having microscopic surface reliefs in a surface thereof. The surface reliefs form optical structures which generate an optical image. The article is made of a cannabis derived resin with cannabinoids selected from the group of THCA (tetrahydrocannabinol acid extracted from trichomes), CBDA and CBGA.

Other advantages of the present invention will become readily apparent from the following detailed description. The invention is capable of other and different embodiments, and its several details are capable of modifications in various obvious respects, all without departing from the invention. Accordingly, the drawing and description are illustrative in nature, not restrictive.

### BRIEF DESCRIPTION OF THE DRAWINGS

The foregoing aspects and many of the attendant advantages of the disclosed embodiments will become more readily appreciated by reference to the following detailed description, when taken in conjunction with the accompanying drawings, wherein:

FIG. 1 depicts a crystalline resin with microscopic surface reliefs, and a production tool for making the surface reliefs in the resin, in accordance with one or more implementations.

FIG. 2 depicts a production tool that may be used to make the crystalline resin shown in FIG. 1, in accordance with one or more implementations.

FIG. 3 depicts a melted resin being applied to a production tool, in accordance with one or more implementations.

FIG. 4 illustrates depicts a melted resin spreading out over a production tool, in accordance with one or more implementations, in accordance with one or more implementations.

FIG. 5 illustrates depicts a non-stick material, being applied over melted resin on a production tool, in accordance with one or more implementations.

FIG. 6 illustrates depicts a roller rolling over the non-stick material, in accordance with one or more implementations.

FIG. 7 illustrates the non-stick material being lifted from the melted resin, in accordance with one or more implementations.

FIG. 8 illustrates the cooled hardened crystalline resin being removed from a production tool, in accordance with one or more implementations.

FIG. 9 illustrates a hardened crystalline resin being broken down into a powder.

FIG. 10 illustrates a hardened crystalline resin after it has been broken down into a powder of micron sized particles.

FIG. 11 illustrates exemplary optical information that can be formed by the surface reliefs at various resolutions.

FIG. 12 illustrates, schematically, a first step in an exemplary process for tagging dry cannabis flower with a powdered resin.

FIG. 13 illustrates, schematically, a second step in an exemplary process for tagging dry cannabis flower with a powdered resin.

FIG. 14 illustrates, schematically, a third step in an exemplary process for tagging dry cannabis flower with a powdered resin.

FIG. 15 illustrates, schematically, various methods in which a tagged dry cannabis flower can be authenticated.

FIG. 16 illustrates and example of a first product that has been tagged by a powdered resin.

FIG. 17 illustrates and example of a second product that has been tagged by a powdered resin.

FIG. 18 illustrates and example of a third product that has been tagged by a powdered resin.

### DETAILED DESCRIPTION

In the following description, for the purposes of explanation, numerous specific details are set forth in order to provide a thorough understanding of the present invention. It will be apparent, however, to one skilled in the art that the present invention may be practiced without these specific details.

One aspect of the invention includes a novel product made of a crystalline resin that has microscopic surface reliefs that reflect or transmit light in a desired pattern. For example, as described in more detail below, light may be reflected or transmitted to generate images with holographic effects. The product may be heated and ingested or consumed through vaporization or by smoking. The product may also be made into other edible products and consumed orally.

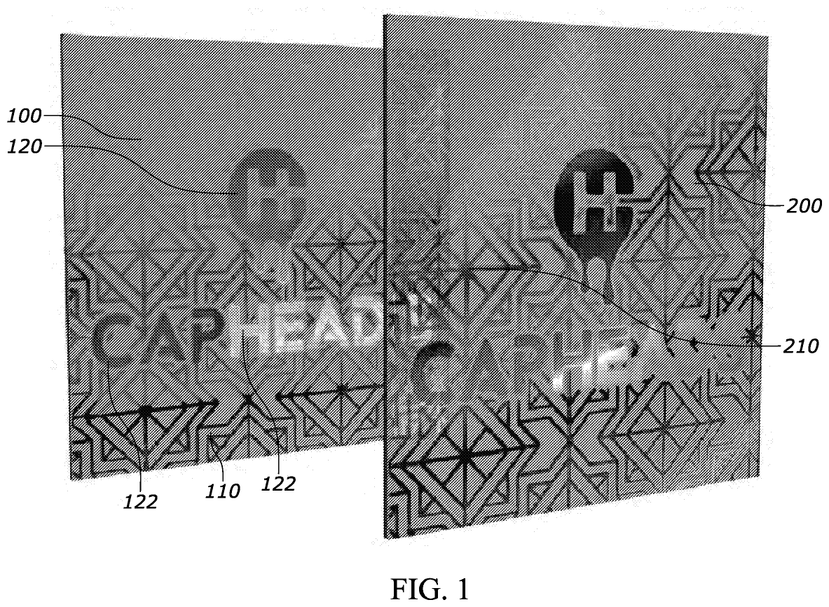

In this aspect, FIG. 1 depicts a cannabis-derived, crystalline resin (100) with microscopic surface reliefs, in accordance with one or more implementations. As shown in FIG. 1, the resin (100) has surface reliefs that are created in one or more predetermined patterns using a production tool (200). Some surface reliefs generate optical images in the form of geometric designs (110) replicated generally across the entire surface of the resin. There are also surface reliefs that generate optical images in the form of logos (120) and letters (122). The surface reliefs may generate images that have various colors.

The crystalline resin (100) product may have microscopic surface reliefs, such as gratings, corresponding to color (pixels) and surface reliefs corresponding to non-color effects (sub-pixels). For example, the pixels may include first pixels corresponding to a first color and second pixels corresponding to a second color. The sub-pixels may include first sub-pixels corresponding to a first non-color effect and second sub-pixels corresponding to a second non-color effect. The surface reliefs in the crystalline resin product may generate images with 2D, 3D or holographic effects.

Preferably, the resin (100) used in this invention is a cannabis-derived resin that can be poured and melted when heated and can crystallize when cooled. The crystalline structure has an index of refraction which helps in refracting or reflecting light from the surface reliefs formed in the resin (100). Examples of resins that may be used include cannabis-derived resins such as those containing cannabinoids such as THCA, CBDA, CBGA or any other cannabinoid that crystallize. For example, such a cannabis-derived resin may be made mostly of isolated cannabinoids, such as THCA (tetrahydrocannabinol acid extracted from trichomes), CBDA (cannabidiolic acid), and CBGA (cannabigerolic acid). But the cannabis-derived resin (100) may contain any other cannabinoid that allow the resin to crystallize or harden. The cannabis-derived resin (100) may also be made of a combination of isolated cannabinoids and other cannabinoids or terpenes that ordinarily do not crystallize or harden on their own, but crystallize when combined in the resin (100).

Generally, cannabis-derived resins are resins derived from the trichomes of a cannabis plant. Cannabis-derived resins include hemp-derived resins (e.g., cannabis with low levels of THC) as they also have a similar physical and chemical make-up and can crystallize as well. The cannabis-derived resin (100) may also be derived from Cannabis ruderalis.

Preferably the cannabis-derived resin (100) does not contain oils or other impurities. Also, preferably, the resin (100) does not contain significant moisture. If the resin (100) contains moisture it may limit the time the impression lasts in the resin (100) as the resin's shape may be more likely to change over time.

The surface reliefs formed in the crystalline resin (100) may reflect or transmit light. The conditions associated with reflection and/or transmission may include conditions related to one or more of viewing angle, viewing distance, polarization, intensity, scattering, refractive index, birefringence, and/or other conditions.

According to some implementations, an optical image may comprise one or more of a hologram, a stereo image, an optically variable device (OVD) based image, a diffractive optically variable image, a zero order device (ZOD) based image, a blazed diffraction structure based image, a first order device (FOZ) based image, a dot matrix image, a pixelgram image, a structural color structure based image, a diffractive identification device (DID) based image, an interference security image structure (ISIS) based image, a kinegram image, an excelgram image, a diffractive optical element based image, a photonic structure based image, a nanohole based image, computer generated holograms, electron-beam generated optical structures, interference patterns, specular and scratch patterns, moire images, light field images, and/or other optical images.

According to some implementations, a person may view the optical image from a specific viewpoint or viewing window (e.g., a range of viewing angles and/or distances). By changing the viewpoint or viewing window (e.g., by moving the optical image relative to the person's eyes), observed colors of the optical image may change due to the reflective properties of the optical structures included in the optical image. The viewpoint or viewing window may be limited in implementations where only the optical structures provide color in the optical image.

According to some implementations, two-dimensional and/or three-dimensional effects may be created in a cannabis-derived crystalline resin (100). A crystalline resin (100) with such surface relief structures may have a variety of applications. For example, optical images may be created in the resin (100) that have visual effects, dynamic patterns, covert and other information.

Some implementations may be used in optical encoding and/or for tracking purposes. In such implementations, the optical images may be one or more of data or codes or other information. Codes may be encrypted or unencrypted.

In some implementations, objects or products may be encoded with optical images. This may provide an extra layer of security due to the fact that the optical images may also have hidden security characteristics. Even without the characteristic of optical hidden security, exemplary embodiments used with encoding offer a layer of security to the object or product.

Some implementations may be used in optical encoding for tracking purposes. For example, surface reliefs may form one or more of optical data or codes. The data or codes may be encrypted or unencrypted. In some implementations, objects or products may be encoded with optical images. This may add an extra layer of security due to the fact that these optical images may also have hidden security characteristics. Even without the characteristic of optical hidden security, exemplary embodiments used with encoding offer a layer of security to the object or product.

Generally, the surface reliefs are formed in the resin (100) using heat and pressure to imprint surface reliefs into the resin (100). As described in more detail below, the desired resin (100) is first heated to a liquid or melted state, in other words heated to a state where it is not crystalline, such that it can be poured and/or imprinted. The heated resin (100) is then poured, injection molded or otherwise transferred onto a production tool (200) such as a shim, film or holographic mold, which itself has surface reliefs that are desired to be transferred to the resin (100). The surface reliefs formed in the resin (100) will be the negative of the surface reliefs in the production tool (200). This method of forming reliefs is sometimes referred to as embossing, engraving and/or casting. The resin (100) is then cooled and crystallizes into a crystalline resin (100) containing the surface reliefs.

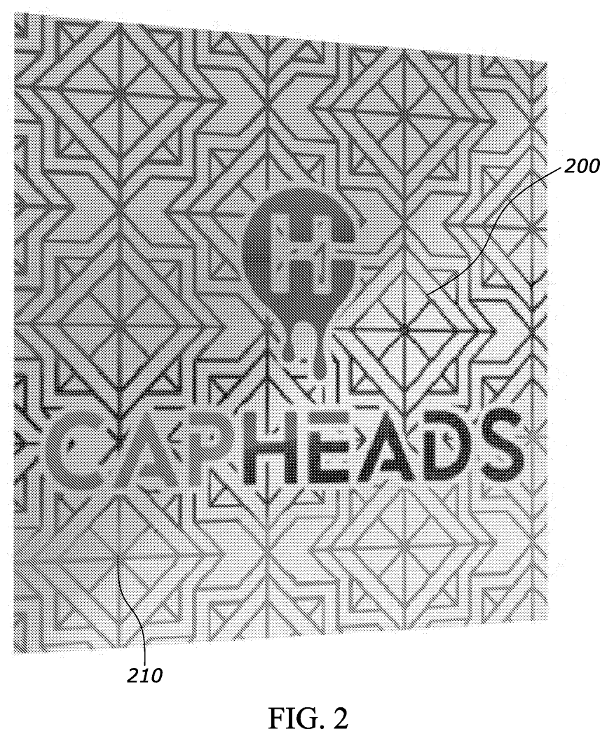

As shown in FIG. 2, a production tool (200), such as a shim will have surface reliefs, such as gratings. The surface reliefs in the production tool (200) may be specially designed and formed in predetermined or desired designs or patterns to create a desired image in the resin. Surface reliefs may have various shapes and sizes to create different visual effects in the resin.

The surface reliefs in the production tool (200) may be microscopic. For example, in some implementations, a given surface relief may have a linear dimension in the range of 0.01 microns to 1000 microns. Similarly, and as a result, a given optical structure formed in the production tool (200) may include a physical feature having a linear dimension in the range of 0.01 microns to 1000 microns.

The production tool (200) may be made of a type of silicone or plastic material. The production tool (200) may also be a plate made of a metal, such as nickel, stainless steel, or aluminum. The production tool (200), such as an engraved injection mold or shim, may be formed from a master tool, using common techniques for making such production tools. For example, surface reliefs may be etched into the master tool by various known processes, including a chemical process, a laser engraving process, nano lithography or an ion etching process. The master tool may be used to create a production tool, such as the production tool (200), directly or indirectly through various other child tools using common techniques. The production tool, such as the production tool (200), will have the same surface reliefs as the master tool or a negative of the surface reliefs in the master tool.

The production tool (200) may also have a holographic design (210) with covert and overt surface relief features such as dynamic effects, security nanotext, micro text, or moire effects. As described below, all of these surface reliefs and features can be transferred to the resin, leaving information embedded in the crystal resin that can be visually appealing as well as providing, though the covert and overt features, security data that can be retrieved by a number of methods and devices, such as microscopes, laser scanning, polarized screens, and Moire decoding film. The production tool (200) may be used to create or replicate surface reliefs in a heated resin that, when cooled, crystallizes and stores the surface reliefs in the hardened resin.

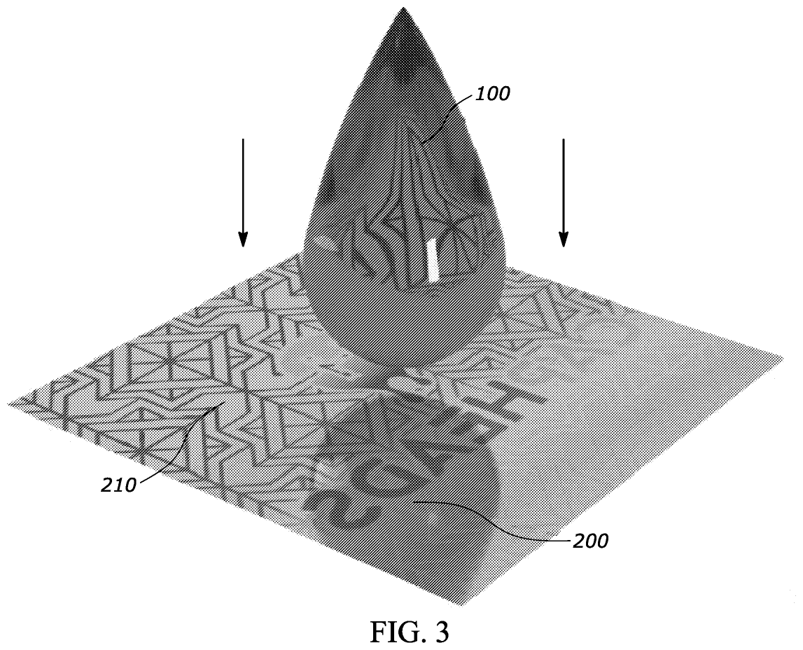

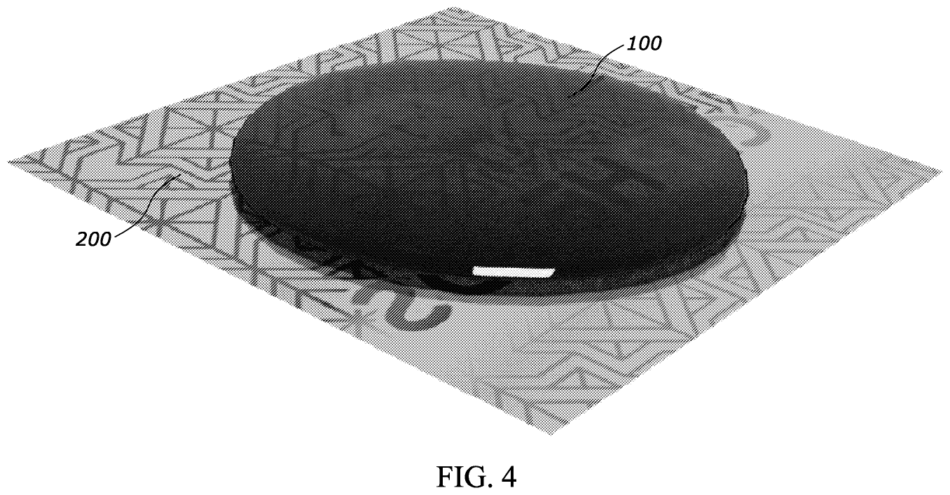

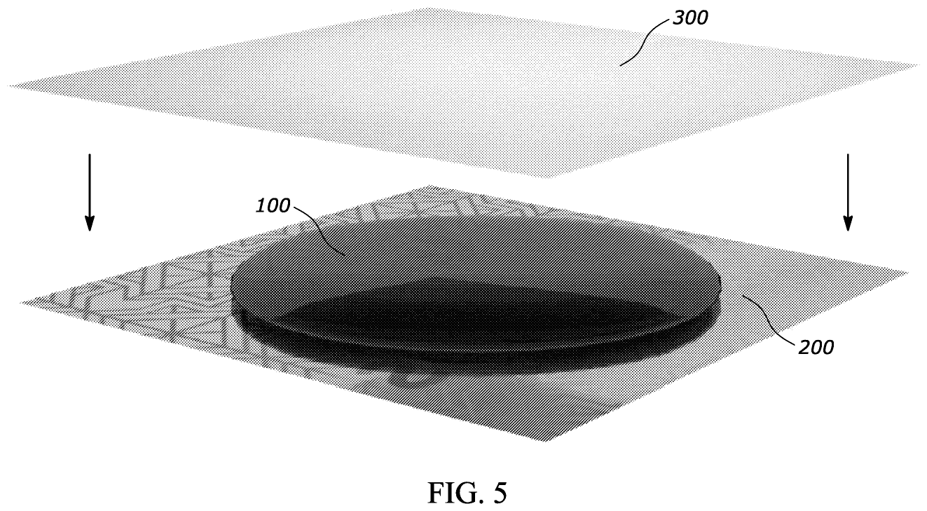

As shown in FIG. 3, a heated or melted resin (100) may be applied to a production tool (200) in accordance with one or more implementations. Heated and melted THCA and/or CBDA resin (100) may be poured on top of the production tool (200). The production tool (200) may be made of silicone, plastic or metal material. As shown in FIG. 4, the heated resin (100) will begin to spread out over the production tool (200) and as shown in FIG. 5, a non-stick material (300), such as parchment paper can be placed over the melted resin (100).

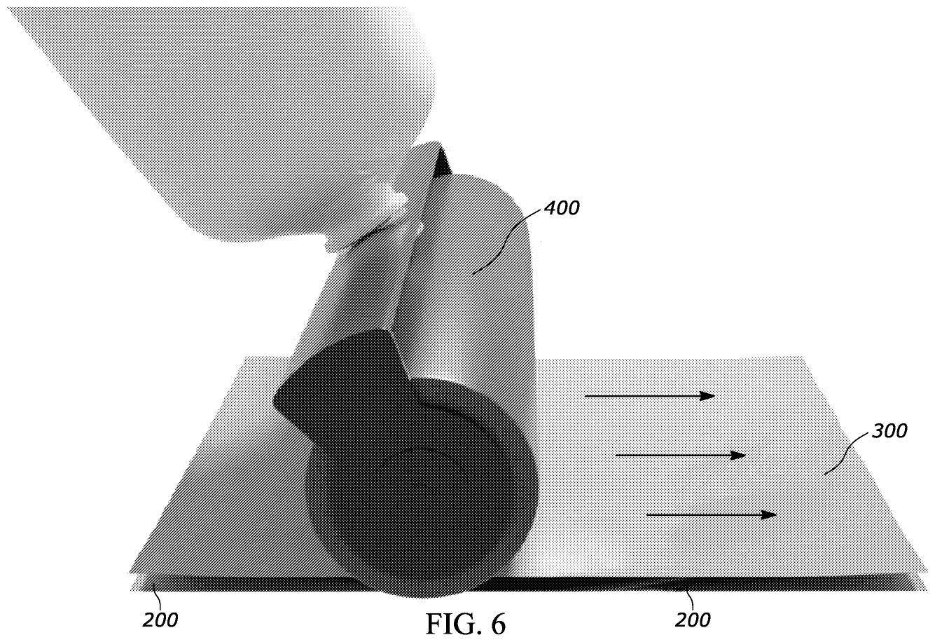

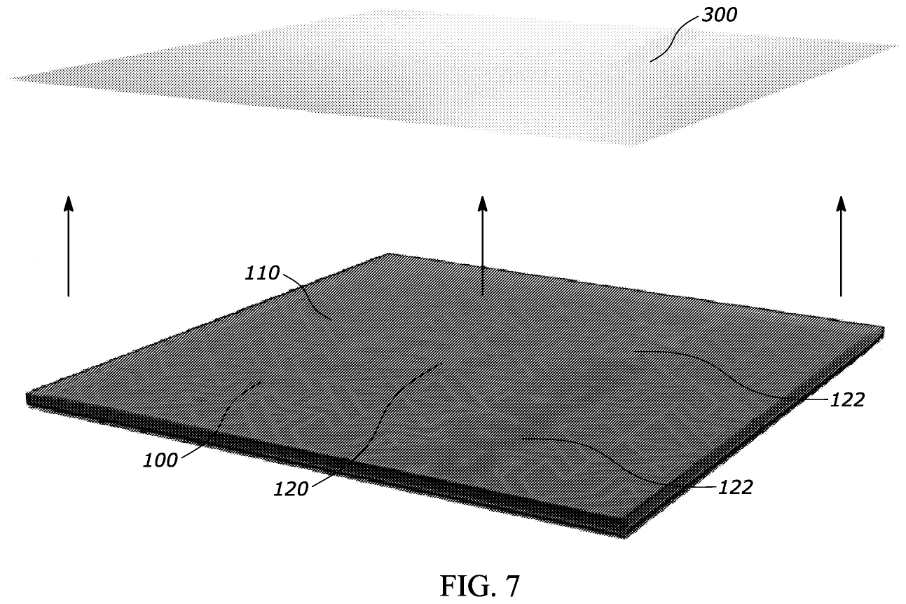

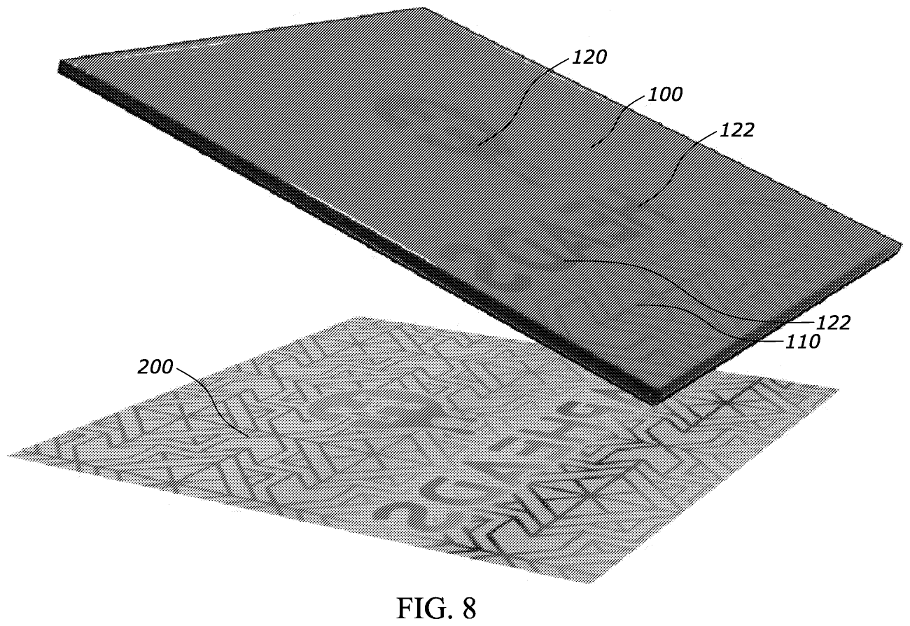

As shown in FIG. 6, another tool such as a rubber roller (400) can used to further and more evenly flatten or spread the resin over the production tool (200) by, for example, rolling the roller (400) over the non-stick material (300). As the resin cools it will harden back to a crystalline state and the surface reliefs will be stored or replicated in the resin. As shown in FIGS. 7 and 8, after the resin has cooled sufficiently, the non-stick material (300) is released or removed from the top of the resin (100). Next, at the appropriate time, e.g., after the resin (100) cools and crystallizes sufficiently, it is separated or removed from the production tool (200). The cooled crystallized or crystalline resin (100) will then contain microscopic surface reliefs which are the negative of those in the production tool (200). The cooled resin (100) is crystallized, and because it cannot be molded in its crystallized form, the surface reliefs will remain in the surface of the crystalline resin (100).

While there may be some degradation of the surface reliefs in the crystalline resin over time, for example, if the product is exposed to prolonged humidity, under normal circumstances, the crystalline resin's surface generally will not deform such that the surface reliefs will remain in the product during the product's normal shelf-life until used by a consumer.

In another example, resin is heated and poured into a first production tool, such as a plain shim or a plain mold (e.g., a mold without surface reliefs). A second production tool with surface reliefs, similar to the production tool described above with respect to FIG. 2, may then be pressed down on top of the heated resin to form surface reliefs. At the appropriate time, e.g., after the resin cools and crystallizes sufficiently, the second production tool is then lifted and the hardened resin containing the surface reliefs on one or both sides is then removed from the first production tool or mold. This method may be used to form surface reliefs in cannabis derived resins or other resins, such as resins used to make hard candy or chocolates.

In another example, resin is heated and poured into a mold that has surface reliefs formed in it. The surface reliefs may be formed into the inner walls of a preformed mold so that, once the resin is cooled and the mold is pulled away, the resin contains the surface reliefs. This may be used to make a capsule made of a cannabis-derived resin with surface reliefs. The surface reliefs may be formed into one or more sides of the resin. For example, surface reliefs may be formed on the front and back sides of the resin. Depending on the shape of the resin and mold, surface reliefs may be formed anywhere on the outside of the resin.

In addition to creating visually appealing designs, the product may be useful for security or forensic purposes as well. For example, the microscopic surface reliefs may be viewed with an electron microscope to see and analyze relief structures in the product. This may be useful for anti-counterfeiting purposes as well as for track and tracing purposes as a unique design or information in the surface reliefs may indicate the product was created by a particular manufacturer or for a particular distributor. Also, if the surface reliefs do not match those of a particular manufacturer or distributor, they may be considered counterfeit and/or not authentic.

The microscopic surface reliefs created in the crystalline resin may vary in size and will correspond to the size of the reliefs in the production tool. For example, in some implementations, surface reliefs may have linear dimensions in the range of 0.01 microns to 1000 microns. Similarly, and as a result, a given optical structure formed in the resin may include a physical feature having a linear dimension in the range of 0.01 microns to 1000 microns.

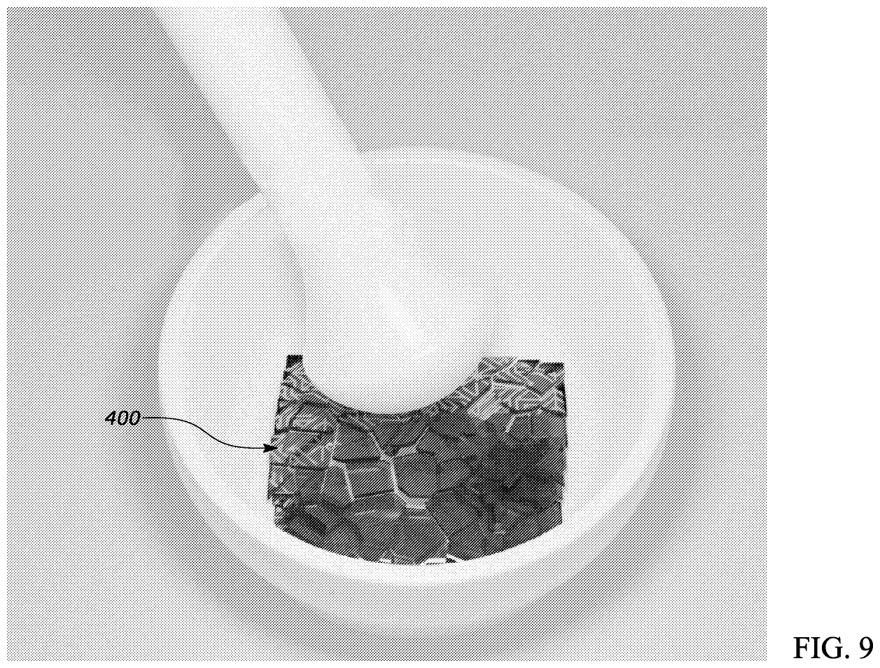

Furthermore, as shown in FIG. 9, the crystalline resin product (100) with the surface reliefs may be pulverized, i.e., broken down into powder, small pieces or particles (400). As shown in FIG. 10, the hardened crystalline resin (200) may be broken down into a powder of micron sized particles (500). The surface relief structures will be present on each piece or particle because the relief structures are smaller than the particles. Thus the pulverized crystalline particles (400 or 500) may still have many relief structures on their surface

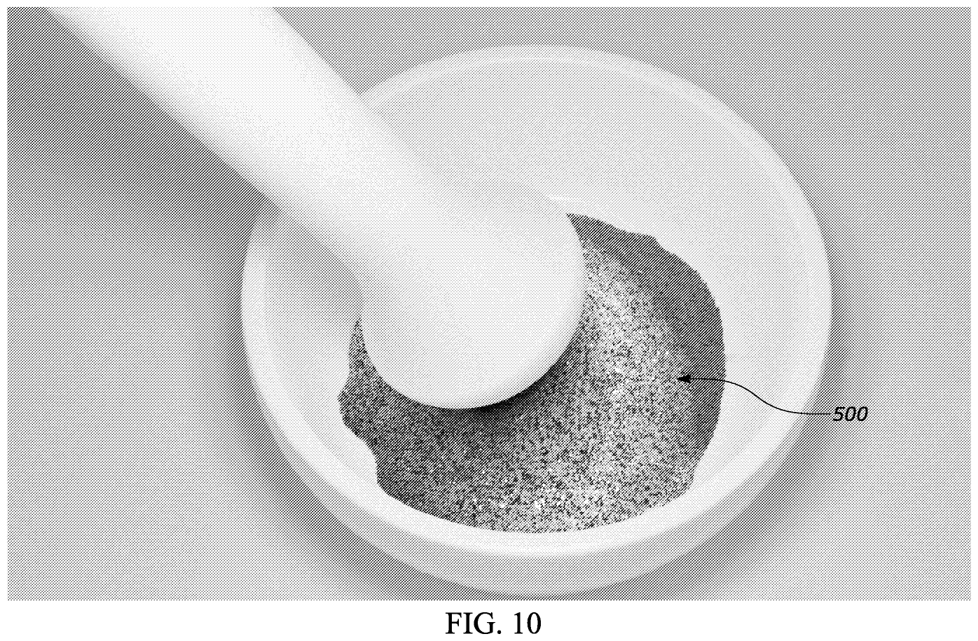

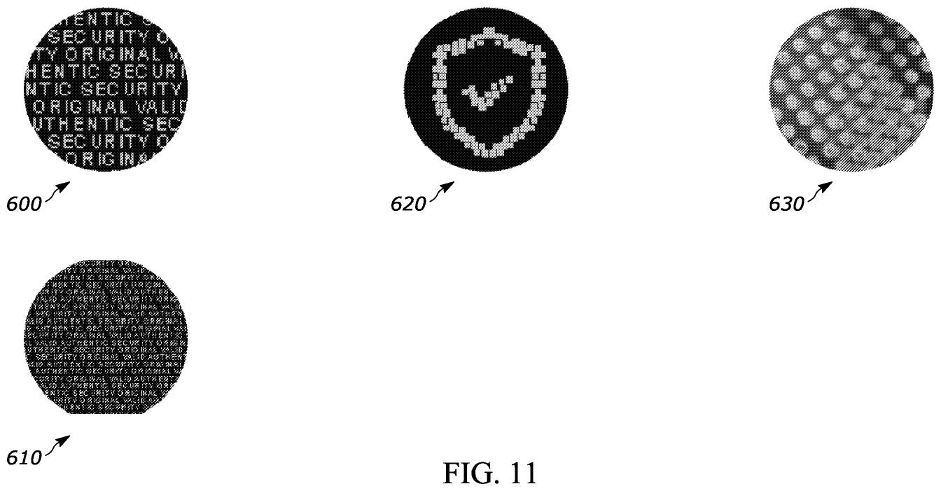

As shown in FIG. 11, different types of optical information can be formed by the surface reliefs depending on the resolution of the surface reliefs. For example, if the particle (600) is 60 microns, optical images forming 12 characters (such as letters or numbers) that are 5 microns in size may be formed on one side of the particle. In another example, if the particle (610) is 200 microns in size, optical images forming 40 characters that are 5 microns in size may be formed on one side of the particle. As noted above, such characters can be made on both sides of the particle. In another example, the particle (620) may contain encrypted data. For example, the particle may contain a double hidden image that hides information that is appears differently when viewed from different angles. This optical image may be, preferably, no smaller than about 80 microns to so to provide a variation of one to two letters or numbers when viewed at different angles. The particle may also contain a hidden image where a laser is needed to view the hidden information. For example, the optical images in this case may be smaller 180×180 microns area is needed. In another example, the particle's image (not shown) may have Moire effects that can be used for forensic analysis and therefore produce hidden images that must be viewed using an appropriate filter. The optical images may be as small as about 250 microns. These hidden images may be viewed using an appropriate filter (i.e., a filter that corresponds to the Moire effect) and using a high power microscope. In another example, if the particle (630) may contain image, such as dots, in a high resolution, e.g., 28,000 dots per inch. Here, the point for each dot would be about 900 nano meters (One microns has=1000 nanometers). By manipulating the surface of each dot other effects that can be created and used for product validation. But validation could also be done by counting the number of dots, i.e., simply by verifying the resolution of the dots. As noted above, the resin can be broken down into other sizes as well, which allows it to be scanned or decoded by a laser or other validating device that can react in a predetermined way based on the known surface relief pattern created in the resin. The pattern may also be decoded on the spot using a microscope that is strong enough to see the patterns. The pulverized crystalline particles also may be sprayed or dusted onto other products.

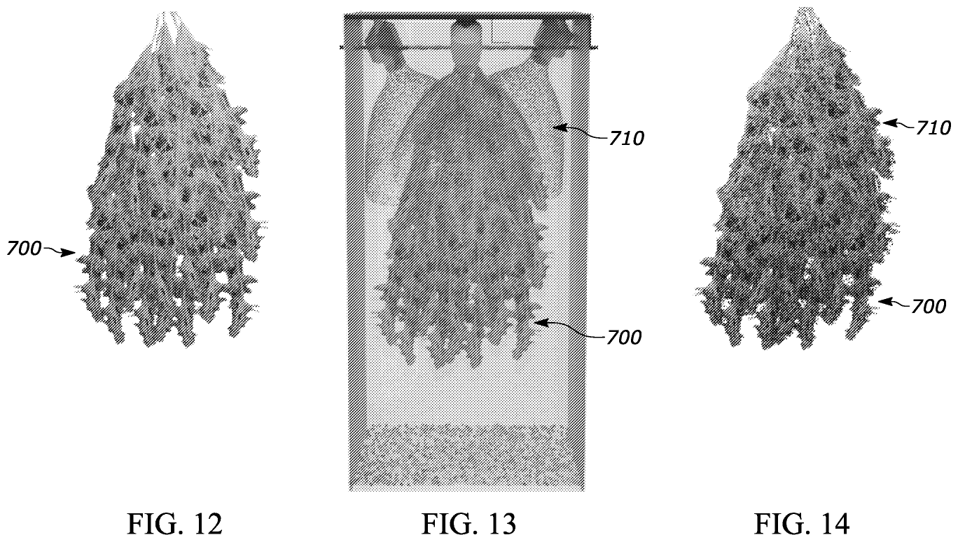

One example of a method and system for decoding such patterns, for example for a tracking and tracing a product using crystalline resin product with the surface reliefs, is shown in FIGS. 12-14. As shown in FIG. 12, a product (700), such as a dried or semi dried cannabis flower bud may be provided. As shown in FIG. 13, particles of the pulverized crystalline resin with the surface reliefs (710) may be sprayed or otherwise applied to the product in a chamber, with some resin particles (710) falling onto the product and some falling to the floor of the chamber. The product falling to the floor may be recycled for further use. As shown in FIG. 14, after the product has been sprayed with resin, numerous micron sized particles, e.g., hundreds of thousands, of the resin remains on the product, which as described below, allows the product to be authenticated. A product that has been sprayed with such resin particles or otherwise has such particles applied thereon may be referred to herein as a tagged product.

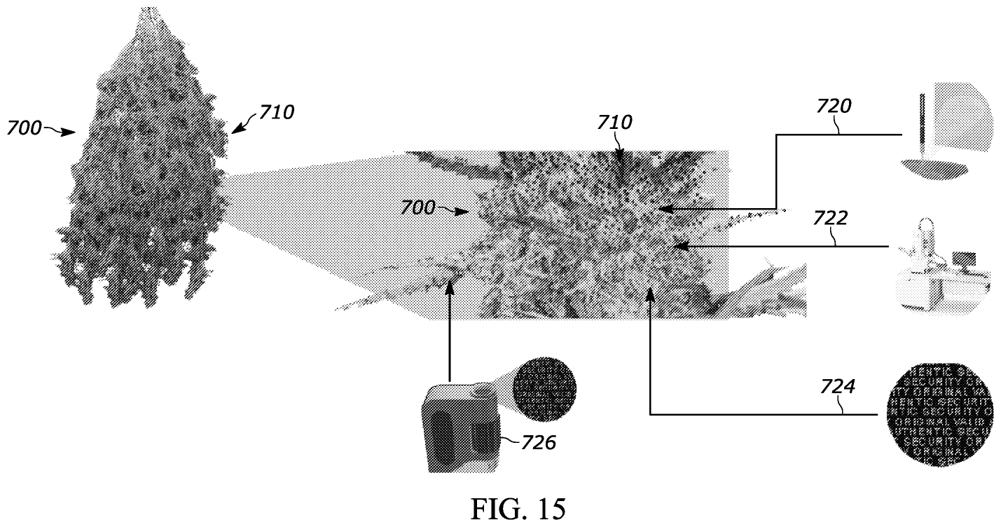

As shown in FIG. 15, the tagged product (700) shown in FIG. 14, for example, may contain pulverized crystalline resin that are about 60 nanometers in size (710). Depending on the size or resolution of the surface reliefs, these particles may contain a hidden image that can be analyzed, such as by being viewed with a laser (720) automatically with an automatic electron digital device or manually. The particles may also contain information that can be analyze by viewing it with a scanning electron microscope (722) and used for forensic analysis. The particles may also contain nanotext information (724) that can be analyzed by viewing it with other equipment, such as a microscope (726). Information about the images that can be formed by the surface reliefs in the resins may be stored in a database. The images analyzed in the particles may be compared with the information in the database. If the analyzed images match information in the database, the product may be verified as authentic, e.g., made by a certain supplier. It may also tell who made the product, when it was made, where it was made, what the product's contents are and any other information that is desired to be tracked and traced. If the analyzed images do not match information in the database, the product may be flagged as not authentic.

In one exemplary method for tracking and tracing a product, a database of known optical images that have been used to make a powder made of numerous micron sized cannabis-derived resin particles can be stored in an electronic storage device. A product containing numerous micron sized cannabis-derived resin particles containing an optical image may be analyzed. Specifically, the particles can be analyzed to determine if they contain an image that matches one of the known optical images in the database. If the images match a known image in the database, then the product may be authenticated. If the images in the powder do not match a known image, the product may not be authenticated.

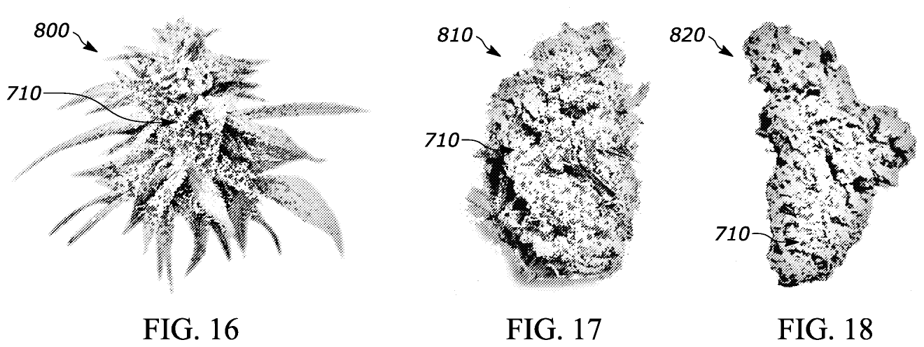

FIGS. 16-18 illustrate various cannabis products that can be tagged with a powdered or pulverized crystalline resin having surface reliefs as described above. FIG. 16 illustrates a first product (800) that has been tagged by a powdered or pulverized crystalline resin. In this example, the product is a fresh cannabis flower. FIG. 17 illustrates and example of a second product (810) that has been tagged by a powdered or pulverized crystalline resin. In this example, the product is a semi-dried cannabis flower. FIG. 18 illustrates and example of a third product (820) that has been tagged by a powdered or pulverized crystalline resin. In this example, the product is a dried cannabis flower. In addition to cannabis flowers, other edible or ingestible cannabis products can be tagged as well. In addition, edible or ingestible non-cannabis products may be tagged as well, such as food or candy.

In a first aspect, an article comprising a crystalized resin material comprising a cannabis derived resin; the material having microscopic surface reliefs in a surface thereof; wherein the surface reliefs form optical structures which generate an optical image and or holographic information.

In a second aspect, the article of aspect 1 wherein the cannabis derived resin is derived from trichomes of a cannabis plant.

In a third aspect, the article of aspect 2 wherein the cannabis derived resin comprises cannabinoids selected from the group of THCA, CBDA and CBGA.

In a fourth aspect the article of aspect 1 wherein the optical structures include one or more of a grating, a hologram, a kinegram, a Fresnel lens, a diffractive optically variable image device, a pixelgram, a holographic stereogram, a diffraction identification device, a photonic structure, a dielectric structure, a volume hologram, an interference security image structure, Photonic Structure a computer-generated hologram, or an electron-beam grating.

In a fifth aspect, the article of aspect 1 wherein the microscopic surface reliefs formed in the resin form optical structures which generate an optical image and or optical information.

In a sixth aspect, a method for fabricating an optical image in a crystalline resin, the method comprising obtaining cannabis-derived resin that can be crystalized; heating the resin; placing the heated resin onto a production tool that has a surface with microscopic surface reliefs; spreading the heated resin over at least part of the surface of the production tool; imprinting surface reliefs in the heated resin; allowing the resin to cool and crystallize; and removing the resin from the production tool after it has crystallized sufficiently.

In a seventh aspect, the method of aspect 6 wherein the microscopic surface reliefs formed in the resin form optical structures which generate an optical image.

In an eighth aspect, the method of aspect 7 wherein the optical structures include one or more of a grating, a hologram, a kinegram, a Fresnel lens, a diffractive optically variable image device, a pixelgram, a holographic stereogram, a diffraction identification device, a Photonic Structure a dielectric structure, a volume hologram, an interference security image structure, a computer-generated hologram, or an electron-beam grating.

In a ninth aspect the method of aspect 8 wherein the resin is derived from trichomes of a cannabis plant.

In a tenth aspect, the method of aspect 9 wherein the cannabis derived resin comprises cannabinoids selected from the group of THCA, CBDA and CBGA.

In an eleventh aspect, a method for tracking and tracing a product, comprising the steps of providing a database containing known optical images; providing a product containing micron sized particles made of cannabis-derived resin; analyzing the particles on the product to determine if they contain an optical image that matches one of the known optical images in the database.

In a twelfth aspect, the method of aspect 11, wherein the particles contain surface reliefs that form optical structures which generate an optical image.

In a thirteenth aspect, the method of aspect 11 wherein the cannabis derived resin is derived from trichomes of a cannabis plant.

In a fourteenth aspect, the method of aspect 12 wherein the cannabis derived resin comprises cannabinoids selected from the group of THCA, CBDA and CBGA.

In a fifteenth aspect, the method of aspect 11 wherein the optical structures include one or more of a grating, a hologram, a kinegram, a Fresnel lens, a diffractive optically variable image device, a pixelgram, a holographic stereogram, a diffraction identification device, a photonic structure, a dielectric structure, a volume hologram, an interference security image structure, Photonic Structure a computer-generated hologram, or an electron-beam grating.

In a sixteenth aspect, the method of aspect 11 wherein the microscopic surface reliefs formed in the resin form optical structures which generate an optical image and or optical information.

In a seventeenth aspect, the method of aspect 11 wherein the particles are analyzed using a microscope.

In an eighteenth aspect, the method of aspect 11 wherein the particles are analyzed using a laser.

In a nineteenth aspect the method of aspect 11 wherein he particles are analyzed using a scanning electron microscope.

In a twentieth aspect a product comprising a first edible or ingestible product and particles made of cannabis-derived resin having surface reliefs applied to the first edible product.

In a twenty-first aspect, the product of aspect 20 wherein the particles made of cannabis-derived resin are edible or ingestible.

In a twenty-second aspect, the product of aspect 20 wherein the particles made of cannabis-derived resin are micron-sized.

In a twenty-second aspect, the product of aspect 20 wherein the particles made of cannabis-derived resin have linear dimensions in the range of 0.01 microns to 1000 microns.

In a twenty-third aspect, the articles of aspects 1 through 5, wherein the crystalized resin material has a linear dimension in the range of 0.01 microns to 1000 microns.

In a twenty-fourth aspect, the methods of aspects 6-10, wherein the crystalized resin material is broken down into particles.

In a twenty-fifth aspect, the methods of aspects 25, wherein the particles have a linear dimension in the range of 0.01 microns to 1000 microns.

In a twenty-sixth aspect, the methods of aspects 11-19, wherein micron-sized particles have a linear dimension in the range of 0.01 microns to 1000 microns.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

The invention claimed is:

1. An article comprising:
a crystalized resin material comprising a cannabis derived resin;
the material having microscopic surface reliefs in a surface thereof;
wherein the surface reliefs form optical structures which generate an optical image and or holographic information.

2. The article of claim 1 wherein:
the cannabis derived resin is derived from trichomes of a cannabis plant.

3. The article of claim 2 wherein:
the cannabis derived resin comprises cannabinoids selected from the group of THCA, CBDA and CBGA.

4. The article of claim 1 wherein:
the optical structures include one or more of a grating, a hologram, a kinegram, a Fresnel lens, a diffractive optically variable image device, a pixelgram, a holographic stereogram, a diffraction identification device, a photonic structure, a dielectric structure, a volume hologram, an interference security image structure, Photonic Structure a computer-generated hologram, or an electron-beam grating.

5. The article of claim 1 wherein:
the microscopic surface reliefs formed in the resin form optical structures which generate an optical image and or optical information.

6. A method for fabricating an optical image in a crystalline resin, the method comprising:
obtaining cannabis-derived resin that can be crystalized;
heating the resin;
placing the heated resin onto a production tool that has a surface with microscopic surface reliefs;
spreading the heated resin over at least part of the surface of the production tool;
imprinting surface reliefs in the heated resin;
allowing the resin to cool and crystallize; and
removing the resin from the production tool after it has crystallized sufficiently.

7. The method of claim 6 wherein:
microscopic surface reliefs formed in the resin form optical structures which generate an optical image.

8. The method of claim 7 wherein:
the optical structures include one or more of a grating, a hologram, a kinegram, a Fresnel lens, a diffractive optically variable image device, a pixelgram, a holographic stereogram, a diffraction identification device, a Photonic Structure a dielectric structure, a volume hologram, an interference security image structure, a computer-generated hologram, or an electron-beam grating.

9. The method of claim 8 wherein:
the resin is derived from trichomes of a cannabis plant.

10. The method of claim 9 wherein:
the cannabis derived resin comprises cannabinoids selected from the group of THCA, CBDA and CBGA.

11. A method for tracking and tracing a product, comprising the steps of:
providing a database containing known optical images;
providing a product containing micron sized particles made of cannabis-derived resin;
analyzing the particles on the product to determine if they contain an optical image that matches one of the known optical images in the database.

12. The method of claim 11, wherein:
the particles contain surface reliefs that form optical structures which generate an optical image.

13. The method of claim 11 wherein:
the cannabis derived resin is derived from trichomes of a cannabis plant.

14. The method of claim 12 wherein:
the cannabis derived resin comprises cannabinoids selected from the group of THCA, CBDA and CBGA.

15. The method of claim 11 wherein:
the optical structures include one or more of a grating, a hologram, a kinegram, a Fresnel lens, a diffractive optically variable image device, a pixelgram, a holographic stereogram, a diffraction identification device, a photonic structure, a dielectric structure, a volume hologram, an interference security image structure, Photonic Structure a computer-generated hologram, or an electron-beam grating.

16. The method of claim 11 wherein:
the microscopic surface reliefs formed in the resin form optical structures which generate an optical image and or optical information.

17. The method of claim 11 wherein:
the particles are analyzed using a microscope.

18. The method of claim 11 wherein:
the particles are analyzed using a laser.

19. The method of claim 11 wherein:
the particles are analyzed using a scanning electron microscope.

20. A product comprising:
a first edible product;
micron sized particles made of cannabis-derived resin having surface reliefs applied to the first edible product.
